(19) United States
(12) Patent Application Publication
(10) Pub. No.: US 2018/0130560 A1
(43) Pub. Date: May 10, 2018

(54) A SYSTEM FOR PATIENT CONTROL AND CARE

(71) Applicant: BYS GRUP BILISIM SISTEMLERI DANISMANLIK TICARET VE SANAYI LIMITED SIRKETI, Golbasi/Ankara (TR)

(72) Inventors: Ahmet Emre OZTUNA, Golbasi/Ankara (TR); Osman Tolga KASKATI, Golbasi/Ankara (US)

(73) Assignee: BYS GRUP BILISIM SISTEMLERI DANISMANLIK TICARET VE SANAYI LIMITED SIRKETI, Golbasi/Ankara (TR)

(21) Appl. No.: 15/569,711

(22) PCT Filed: Apr. 29, 2016
(86) PCT No.: PCT/TR2016/000060

§ 371 Date: Oct. 26, 2017

(30) Foreign Application Priority Data
Apr. 30, 2015 (TR) 2015/05285

(51) Int. Cl. G16H 80/00; G16H 10/00

(52) U.S. Cl. CPC G16H 80/00; G16H 10/00

(57) ABSTRACT

The present invention relates to a remote patient control and care system (1) which is developed for facilitating the control and care of particularly Alzheimer patients and elderly people. The said system (1) comprises a smart device (2) which is used by the patient (H) and the caregiver (B); a central server (3) which is in communication with the smart device (2); a central database (4) which is managed by the central server (3); a client device (5) which is used by the healthcare personnel (S), can connect to the central server (3) and thus access the central database (4) and is in communication with the smart device (2); and an application server (6) which enables to manage capabilities of the smart device (2) and transactions to be carried out on the smart device (2).

### TECHNICAL FIELD

The present invention relates to a remote patient control and care system which is developed for facilitating the control and care of particularly Alzheimer patients and elderly people.

### BACKGROUND OF THE INVENTION

Today, helping to carry out many transactions by remote access or not has been possible upon increase of prevalence of communication technologies and smart devices enabling to use communication technologies in an effective manner. Transactions of patient control and care are the most important ones among these transactions.

In the state of the art, patients who developed particularly Alzheimer disease or developed mental deficiency disease or mental performance decrease in any manner need to talk with doctor and caregiver face to face in their control and care by conventional methods and it is enabled to carry out evaluation by doctor about the patient's condition by making the patient to play various games or to solve tests in this face to face talk. In patient care with conventional methods, drug use is realized in following ways such as the doctor notifies the caregiver about medications to be used and how to use them verbally or in written and then hand over all responsibility about how to use and follow-up of medication use to the caregiver. These conventional methods in the state of the art also cause problems such as the patient has to talk with doctor or any healthcare personnel face to face every time, games and test cannot be carried out with sufficient efficiency every time in these face to face talks, it is not always possible for a caregiver to remember doctor's appointments and medication use and to practice medication use in a correct way.

Today, there are also some studies which aim to provide patient control and care with a more systematic structure by use of some smart devices other than these conventional methods. Some of these studies are the ones which enable to set a medication reminder time over smart mobile devices and provide games that can be played by patients who developed particularly Alzheimer disease or developed mental deficiency disease or mental performance decrease in any manner, for exercise purpose. However, these studies still require a manual follow-up or don't store patient's performance data related to the games and the tests or don't allow remote control of these data and thus the patient by storing them only on the smart device locally.

Given this system and studies existing in the state of the art and aiming patient control and care, it is seen that there is a need for a system wherein games and test are presented to a patient over a smart device; opportunities such as input of medication use information, reminder are provided to a caregiver over a smart device; maintenance and control of a smart device are made remotely; data related to each transaction carried out on a smart device is gathered in a central server and thus which enables a healthcare personnel to connect this central server to be able to see and control all information about the transactions carried out by a patient or caregiver and thereby which will enable to carry out a patient control and patient care transaction completely remotely for patients who developed particularly Alzheimer disease or developed mental deficiency disease or mental performance decrease in any manner.

The United States patent document no. US20080070207, an application in the state of the art, discloses a system and method which enables to conduct various perceptual and mental memory tests over a mobile device; to store the data (for example a score) generated during conduction of these memory tests; and to send it to a remote server over a communication network.

The United States patent document no. US20140114889, another application in the state of the art, discloses a system and method which enables to carry out mental and perceptual assessment about a person based on the person's mobile device usage.

The European patent document no. EP2660745, an application in the state of the art, discloses a system and method which enables to monitor a user's mental health by tracking a user's use of electronic devices such as usage of mobile phone, tablet and web activity of a user.

### SUMMARY OF THE INVENTION

An objective of the present invention is to realize a patient control and care system wherein time, score, performance and usage data occurring as a result of interactions of Alzheimer patients and elderly people with games and tests shown to them over a smart device are transferred to a central server and these data can be controlled by a healthcare personnel over this central server and thus a remote patient control is enabled.

Another objective of the present invention is to realize a patient control and care system whereby persons taking responsibility for nursing Alzheimer patients and elderly people can enter data about the patient and themselves; input medication information, reminder records; authorize the healthcare personnel who will take care of the patient; watch various training videos about patient care.

Another objective of the present invention is to realize a patient control and care system which enables to make notification or reminder to the caregiver over a smart device according to the information input by the caregiver and/or determined by the central server and/or the healthcare personnel.

Another objective of the present invention is to realize a patient control and care system wherein written, voice and visual communication can be provided among patient and/or caregiver and healthcare personnel remotely.

Another objective of the present invention is to realize a patient control and care system wherein it is enabled make controls for using correct medicine by the central server automatically or to make an examination by a healthcare personnel and to send warning notification to a smart device in case of detecting a non-conformity according to the medication usage data input by the caregiver.

Another objective of the present invention is to realize a patient control and care system wherein management of capabilities of smart device used by patient and caregiver and transactions which can be carried out on this smart device are realized by an application server and thus it is not necessary anymore for patient and caregiver to deal with complex structures of smart devices or features thereof which will disrupt their operation.

### DETAILED DESCRIPTION OF THE INVENTION

“A System for Patient Control and Care” realized to fulfil the objectives of the present invention is shown in the FIGURE attached, in which:

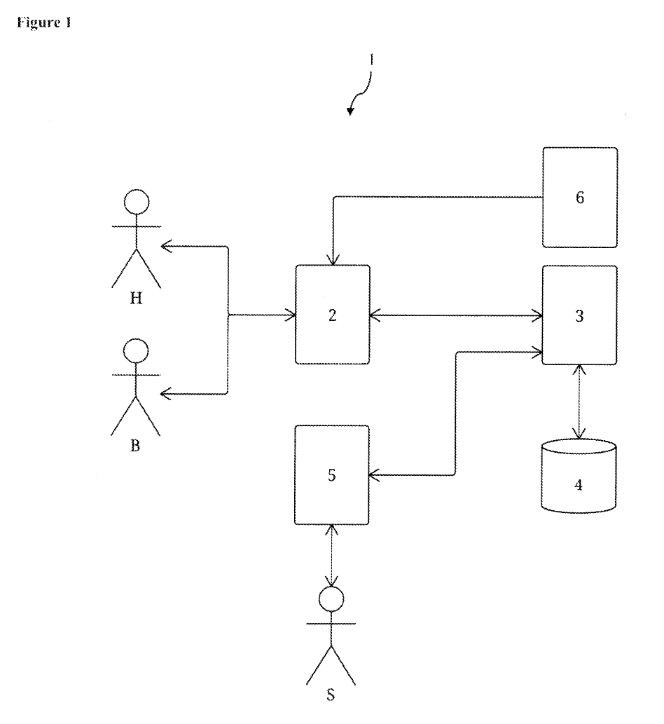

FIG. 1 is a schematic block diagram of the inventive system.

The components illustrated in the FIGURE are individually numbered, where the numbers refer to the following:

1. System

2. Smart device

3. Central server

4. Central database

5. Client device

6. Application server

I. Patient

B. Caregiver

S. Healthcare personnel

A system for patient control and system (1) comprises:
at least one smart device (2) which is used by the patient (H) and the caregiver (B) for accessing games and test, inputting data, inputting reminder record and managing medication usage;
at least one central server (3) wherein the data about the transactions carried out by the patient (H) and the caregiver (B) on the smart device (2) are transferred and which enables to create, display and manage records in accordance with these data;
at least one central database (4) wherein the data reaching the central server (3) and the records created by the central server (3) are stored; and which is managed by the central server (3);
at least one client device (5) which enables the healthcare personnel (S) to connect the central server (3) and to access the data on the central database (4) by means of the central server (3); to manage these data; to communicate with the smart device (2);
at least one application server (6) which enables to manage capabilities of the smart device (2) and updates and settings related to the smart device (2) and which is in communication with the smart device (2) for this purpose (FIG. 1).

The smart device (2) is a device which is used by for accessing games and test, inputting data, inputting reminder record and managing medication usage. In a preferred embodiment of the invention, the smart device (2) is a tablet computer. In other embodiments of the invention, the smart device (2) can be a smart phone or a smart television.

In one embodiment of the invention, voice and visual communication can be provided between the smart device (2) and the client device (5), in other words among the patient (1I) or the caregiver (B) and the healthcare personnel (S).

Therefore, in one embodiment of the invention, the smart device (2) is a device which has camera, microphone and speaker.

The smart device (2) is a device which is in communication with both the central server (3) and the application server (6), and it has a structure and capability to be able to connect mobile or fixed networks enabling to establish communication with the central server (3) and the application server (6).

The smart device (2) comprises a screen which enables to perform views related to the transactions to be carried out by the patient (H) or the caregiver (B), the communication to be established between the client device (5) and the smart device (2), the notifications and the reminders to be made to the patient (H) or the caregiver (B) over the smart device (2). This screen can be a touchscreen in cases where the smart device (2) is a tablet computer and it can serve as an input unit in transactions to be carried out by the patient (H) or the caregiver (B) with this characteristic thereof. In different embodiments of the invention, the input unit owned by the smart device (2) can be a unit comprising various keypads and sometimes in the form of a separate unit (for example, a remote control for a smart television).

The smart device (2) is a device wherein games and tests designed in accordance with patients are displayed to the patient (H) over a graphic interface and which enables to receive interactions of the patients (H) about these games and tests by means of the input unit. The said games and tests are displayed on the screen of the smart device (2) and these games and tests are the ones which are aimed for measuring language, attention, arithmetic, object recognition, memory improvement and visual-spatial functions of patients (H). In an exemplary embodiment of the invention, these games and tests may be in the form of:
memory games, picture and word games for improving memory;
jigsaw game and colour trail game for improving attention;
arithmetic game for improving arithmetic skills;
drawing game for improving configuration skills;
place and time-related games for reducing time and place forgetfulness.

In an exemplary embodiment of the invention, the said games and tests are specified for the case wherein the patient (H) is an Alzheimer patient or an old individual with a high Alzheimer risk; the games and the tests displayed to the patient (H) over the smart device (2) and enables to provide interaction of the patient (H) can be changed and diversified according to various other disease types and different purposes in accordance with these disease types. The smart device (2) is a device which transmits information comprising score, time, performance and usage data such as answers given by the patients (H), time of answering, number of correct answers, number of wrong answers to the central server (3) over an network connection after these games and tests. In a preferred embodiment of the invention, these games and tests displayed to the patient (H) by the smart device (2) are the ones which are levelled according to the difficulty conditions and the patient (H) displays are performed by taking this level information into consideration.

Transaction of managing games and tests to be displayed to the patient (H) over the smart device (2) (such as which gains and test will be displayed, which ones will be added, which ones will be removed) is carried out by the application server (6).

The smart device (2) is a device which provides related graphic interfaces in order to carry out transactions such as inputting information of the caregiver (B), the patient (H) and the healthcare personnel (S); creating record of doctor's appointments and social activity reminder; making entries related to the medication type, the usage chart used by the patient (H) and the usage condition; authorizing the healthcare personnel (S) who will take care of the patient (H). In one embodiment of the invention, the smart device (2) is a device which is configured so as to display training videos about the disease and the patient care to the caregiver (B). The smart device (2) is a device which can store the said video contents locally or receive them from the central database (4) by means of the central server (3).

In one exemplary embodiment of the invention, the graphic interfaces provided to the caregiver (B) by the smart device (2) are presented by headings such as Medication Reminder. Event Reminder. Form Application and Health Personnel Management.

In one embodiment of the invention, after the transactions of creating reminder record and inputting data related to the medications used by the patient (H), which are carried out by the caregiver (B), these records and information are stored by the smart device (2) and these records and information (for example, reminding time for medication usage, reminding an activity, reminding a work from a to-do-list) are controlled by the smart device (2) and then the related notifications can be locally created by the smart device (2) again. In another embodiment of the invention, these records and information are transmitted to the central server (3) by the smart device (2) as the caregiver (B) are making the entries and notifications related to these records and information are (for example, reminding time for medication usage, reminding an activity, reminding a work from a to-do-list) transmitted to the smart device (2) over the central server (3) preferably with push notification method upon their controls are made over the central server (3).

The smart device (2) is a device which is configured such that the healthcare personnel (S) descriptive information that is to be used in the transaction of authorizing the healthcare personnel (S) who will take care of the patient (H) carried out by the caregiver (B), and the password information that is required to be entered by the healthcare personnel (S) are input by the caregiver (B) and these information are transmitted to the central server (3).

The smart device (2) is a device which listens, catches and carries out the commands sent by the application server (6). The smart device (2) is a device which comprises:
command listening;
device limiting;
service loading/installing;
device information

modules in order to listen, catch and carry out the commands sent by the application server (6).

The smart device (2) catches this command by the command listening module when a command requiring device limitation is sent by the application server (6) and prevents the smart device (2) from being used except the transactions that can be carried out by the patient (H) and the caregiver (B) by means of the device limiting module. The device limiting module also enables the smart device (2) to determine whether the patient (H) or the caregiver (B) uses the limited features or not. By means of the device limiting module, the smart device (2) can display a main page —wherein there are only transactions which can be carried out by them—to the patient (H) and the caregiver (B).

The smart device (2) also includes a menu which is protected by a certain password on the main page displayed to the patient (H) and the caregiver (B) by means of the device limiting module and which enables to inactivate the device limiting module; to put wireless network barrier. Bluetooth barrier, flight mode barrier, mobile data network barrier for the smart device (2); to display services and applications that can be seen by the users entering the password. The password enabling entrance to the said menu is also located in the memory within the smart device (2). This password, this menu and the settings about the menu can be set and updated by the smart device (2) via the commands sent over the application server (6).

The smart device (2) is a unit which can make setting and update according to the commands received from the application server (6) related to the transactions that can be carried out by the patient (1H) and the caregiver (B) by means of the service loading/installing module.

The central server (3) is a server whereby usage, record and reminder data about the transactions carried out by the patient (H) and the caregiver (B) on the smart device (2) are transferred and which enables to create, display and manage records in accordance with these data.

The central server (3) is a server which can be accessed by the healthcare personnel (S) by means of the client device (5) as well and whereby s/he can view and set the data located in the central database (4) over thereof. The central server (3) is a server which can be accessed by the healthcare personnel (S) by means of the client device (5) and whereby s/he accesses the data located in the central database (4) over thereof and wherein the patient's (H) score, time, usage data related to the games and the tests are located. The healthcare personnel (S) can make an inference about the patient's (H) condition by evaluating these data.

The central server (3) is also a server which enables the healthcare personnel (S) to establish written, voice and visual communication with the patient (H) and the caregiver (B).

The central server (3) is a unit which controls whether the healthcare personnel (S) has the data that s/he wants to access, view and set in the central database (4) or the necessary authorization about the patient (H) and the caregiver (B) with whom s/he wants to communicate. In one embodiment of the invention, it is controlled by the central server (3) whether the healthcare personnel (S) descriptive information—which is matched with the patient (H) information entered by the caregiver (B) previously for controlling this authorization transaction—and the password information—which is required to be entered by the healthcare personnel (S)—are same as the ones entered by the healthcare personnel (S) by means of the client device (5) and if such control works out, the central database (4) enables the healthcare personnel (S) to access the data for that patient (H) and to communicate with the smart device (2) being used by that patient (H).

In one embodiment of the invention, the central server (3) is a server which is configured such that it will be able to control the entries—which are made by the caregiver (B) by means of the smart device (2) and related to the type and the usage chart of the medications used by the patient (H)—automatically according to the information of medication unconformity and suitable usage chart included in the central database (4); and to send notification to the smart device (2) and/or the client device (5) in case of detecting an unconformity in the medication types related to the patient (H) or among the medications or detecting an unconformity in the control transaction according to the usage chart after this control.

In one embodiment of the invention, the central server (3) is a server which is configured such that it will be able to send medication reminders to the smart device (2) even if no data entry is made related to any medication type or a usage chart by the caregiver (B) by means of the smart device (2) based on the information of reference medication usage included in the central database (4) and prepared particularly for the patient (H) or the disease.

In one embodiment of the invention, the central server (3) is a server which is configured such that it will be able to send a reminder notification to the smart device (2) such that it also comprises the type and the level information of the game and the test, in cases where it is required to apply games and tests based on the information that are prepared previously, preferably by the healthcare personnel (S) about which games and tests should be applied to the patient (H) according to a what kind of timetable on the basis of the patient (H) and/or the disease and added to the central database (4).

The central server (3) is a server wherein the web-based application enabling the healthcare personnel (S) to carry out all kinds of above-mentioned transactions by accessing over the client device (5) is installed. In one preferred embodiment of the invention, the central server (3) is a server which is configured such that it will be able to establish voice or video communication over webRTC technology between the smart device (2) and the client device (5).

The central database (4) wherein the data reaching the central server (3) and the records created by the central server (3) are stored; and which is managed by the central server (3).

In one preferred embodiment of the invention, in the central database (4), there are information of score, time, usage form related to the game and test transactions carried out by the patient (H) by means of the smart device (2); information for transactions of entering information about the patient (H) and the caregiver (B) carried out by the caregiver (B), creating a reminder record for appointment and social activity, making entry related to the type and the usage chart of the medications used by the patient (H), authorizing the healthcare personnel (S) who will take care of the patient (H); information about the patient (H), the caregiver (B), the smart device (2) and the healthcare personnel (S) which are separately descriptive and matched with each other; a password information based on the patient (H) about the healthcare personnel (S). In one embodiment of the invention, in the central database (4), there are also usage chart data of medication conformity and unconformity based on the patient (H) or the disease.

The client device (5) is a unit which enables the healthcare personnel (S) to connect the central server (3) and to access the data on the central database (4) by means of the central server (3); to manage these data; to communicate with the smart device (2).

In one embodiment of the invention, the client device (5) is a computer. In different embodiments of the invention, the client device (5) can be a smart phone or tablet computer which can provide access to the central server (3) and has capability to display the related graphic interfaces with respect to the transactions that can be carried out by the healthcare personnel (S).

The client device (5) is a unit which provides graphic interfaces—wherein transactions of: accessing the central server (3); viewing the data of patient (H) based game and test score, time, performance and usage data, the patient (1H) information, the activity reminder records, the medication type and the usage chart information located in the central database (4) that is accessed by means of the central server (3); sending information and reminder notification (for example reminder notification for doctor's appointment) to the smart device (2) so as to be transmitted to the patient (H) and the caregiver (B); establishing written, voice and visual communication with the patient (H) and/or the caregiver (B) by means of the smart device (2)—can be carried out to the healthcare personnel (S).

In one embodiment of the invention, the client device (5) is a device which can also be used for providing voice and visual communication among the healthcare personnel (S) and the patient (H) and/or the caregiver (B) and thus it is a device comprising camera, microphone and speaker.

The application server (6) is a server which enables to manage capabilities of the smart device (2) and updates and settings related to the smart device (2) and it is in communication with the smart device (2) for this purpose.

The application server (6) enables to send device limiting commands and settings to the smart device (2) so as not to carry out any transaction except the transactions to be carried out by the patient (H) and the caregiver (B) on the smart device (2) in the management of the smart device (2) capabilities. The application server (6) thus enables display and operation of only the graphical interfaces and the services related to the transactions to be carried out by the patient (H) and the caregiver (B) on the smart device (2) independently of the operating system provided on the smart device (2). Removing any device limiting feature on any smart device (2) is also within the control of the application server (6).

The application server (6) is a unit which also carries out the transactions of setting (adding, removing) and updating transactions related to the transactions to be carried out by the patient (1H) and the caregiver (B) on the smart device (2), by means of the commands sent to the smart device (2). This command communication between the application server (6) and the smart device (2) is provided over a cloud messaging service. On the smart device (2), there are command listening, device limiting, service loading/installing, device information modules which listen, catch and carry out these commands sent by the application server (6).

In a preferred embodiment of the invention, the application server (6) is a server which provides an interface for creating commands and settings to be sent to the smart device (2) and for sending these commands and settings.

With the inventive system (1), transactions of: transferring time, score, performance and usage data generated as a result of interactions of Alzheimer patients and elderly people with games and tests shown to them over a smart device to a central server and controlling these data by a healthcare personnel over this central server and thus making remote patient control are carried out.

With the inventive system (1), persons taking responsibility for nursing Alzheimer patients and elderly people are enabled to enter data about the patient and themselves; input medication information, reminder records; authorize the healthcare personnel who will take care of the patient; watch various training videos about patient care.

With the inventive system (1), transaction of making notification or reminder to the caregiver over a smart device according to the information input by the caregiver and/or determined by the central server and/or the healthcare personnel is carried out.

With the inventive system (1), transaction of realizing a remote written, voice and visual communication among patient and/or caregiver and healthcare personnel is enabled.

With the inventive system (1), transactions of making controls for using correct medicine by the central server automatically or to make an examination by a healthcare personnel and to send warning notification to a smart device in case of detecting a nonconformity according to the information of medication usage input by the caregiver are carried out.

With the inventive system (1), it is ensured that management of capabilities of smart device used by patient and caregiver and transactions which can be carried out on this smart device are realized by an application server and thus it is not necessary anymore for patient and caregiver to deal with complex structures of smart devices or features thereof which will disrupt their operation.

It is possible to develop a great variety of embodiments of the inventive system (1); it cannot be limited to the examples disclosed herein and it is essentially according to the claims.

1. A system (1) for patient control and system; comprising:
at least one smart device (2) which is used by the patient (H) and the caregiver (B);
at least one central server (3) wherein the data about the transactions carried out by the patient (H) and the caregiver (B) on the smart device (2) are transferred;
at least one central database (4) wherein the data reaching the central server (3) and the records created by the central server (3) are stored; and which is managed by the central server (3);
at least one client device (5) which enables the healthcare personnel (S) to connect the central server (3) and to access the data on the central database (4) by means of the central server (3); to manage these data; to communicate with the smart device (2);
at least one application server (6) which enables to manage capabilities of the smart device (2) and updates and settings related to the smart device (2) and which is in communication with the smart device (2) for this purpose;
and characterized by:
at least one smart device (2) which is used by the patient (H) and the caregiver (B) for accessing games and test, inputting data, inputting reminder record and managing medication usage;
at least one central server (3) wherein the data about the transactions carried out by the patient (H) and the caregiver (B) on the smart device (2) are transferred and which enables to create, display and manage records in accordance with these data.

2. A system (1) according to claim 1, characterized by the smart device (2) which is a tablet computer.

3. A system (1) according to claim 1, characterized by the smart device (2) which is a smart phone.

4. A system (1) according to claim 1, characterized by the smart device (2) which is a smart television.

5. A system (1) according to claim 1, characterized by the smart device (2) which has camera, microphone and speaker.

6. A system (1) according to claim 1, characterized by the smart device (2) which is a device that has a structure and capability to be able to connect mobile or fixed networks enabling to establish communication with the central server (3) and the application server (6).

7. A system (1) according to claim 1, characterized by the smart device (2) which comprises a screen that enables to perform views related to the transactions to be carried out by the patient (H) or the caregiver (B), the communication to be established between the client device (5) and the smart device (2), the notifications and the reminders to be made to the patient (H) or the caregiver (B) over the smart device (2).

8. A system (1) according to claim 7, characterized by the smart device (2) which has a touchscreen that can serve as an input unit in transactions to be carried out by the patient (H) or the caregiver (B).

9. A system (1) according to claim 1, characterized by the smart device (2) which has an input unit comprising various keypads.

10. A system (1) according to claim 1, characterized by the smart device (2) wherein games and tests designed in accordance with patients are displayed to the patient (H) over a graphic interface and interactions of the patients (H) about these games and tests are received by means of the input unit.

11. A system (1) according to claim 1, characterized by the smart device (2) wherein games for measuring language, attention, arithmetic, object recognition, memory improvement and visual-spatial functions of patients (H) are displayed and played by the patient (H).

12. A system (1) according to claim 1, characterized by a smart device (2) wherein
memory games, picture and word games for improving memory;
jigsaw game and color trail game for improving attention;
arithmetic game for improving arithmetic skills;
drawing game for improving configuration skills;
place and time-related games for reducing time and place forgetfulness are displayed and played by the patient (H).

13. A system (1) according to claim 1, characterized by the smart device (2) which transmits information comprising score, time, performance and usage data such as answers given by the patients (H), time of answering, number of correct answers, number of wrong answers to the central server (3) over an network connection after these games and tests.

14. A system (1) according to claim 1, characterized by the smart device (2) which is a device that provides related graphic interfaces in order to carry out transactions such as inputting information of the caregiver (B), the patient (H) and the healthcare personnel (S); creating record of doctor's appointments and social activity reminder; making entries related to the medication type, the usage chart used by the patient (H) and the usage condition; authorizing the healthcare personnel (S) who will take care of the patient (H).

15. A system (1) according to claim 14, characterized by the smart device (2) which displays the graphic interfaces to the caregiver (B) by headings such as Medication Reminder, Event Reminder. Form Application and Health Personnel Management.

16. A system (1) according to claim 14, characterized by the smart device (2) which—after the transactions of creating reminder record and inputting data related to the medications used by the patient (H) that are carried out by the caregiver (B)—stores these records and information locally and creates the related notifications locally again by controlling these records and notifications.

17. A system (1) according to claim 14, characterized by the smart device (2) which—after the transactions of creating reminder record and inputting data related to the medications used by the patient (H) that are carried out by the caregiver (B)—transmits these records and information to the central server (3) and displays the notifications related to these records and information by receiving them over the central server (3).

18. A system (1) according to claim 14, characterized by the smart device (2) which is configured such that the healthcare personnel (S) descriptive information that is to be used in the transaction of authorizing the healthcare personnel (S) who will take care of the patient (H) carried out by the caregiver (B) and the password information that is required to be entered by the healthcare personnel (S) are input by the caregiver (B) and these information are transmitted to the central server (3).

19. A system (1) according to claim 1, characterized by the smart device (2) which listens, catches and carries out the commands sent by the application server (6).

20. A system (1) according to claim 19, characterized by the smart device (2) which comprises command listening; device limiting; service loading/installing; device information modules in order to listen, catch and carry out the commands sent by the application server (6).

21. A system (1) according to claim 20, characterized by the smart device (2) which is configured to catch this command by the command listening module when a command requiring device limitation is sent by the application server (6) and prevent the smart device (2) from being used except the transactions that can be carried out by the patient (H) and the caregiver (B) by means of the device limiting module.

22. A system (1) according to claim 21, characterized by the smart device (2) which displays a main page—wherein there are only transactions which can be carried out by them—to the patient (H) and the caregiver (B).

23. A system (1) according to claim 22, characterized by the smart device (2) which also includes a menu that is protected by a certain password on the main page displayed to the patient (H) and the caregiver (B) by means of the device limiting module and that enables to inactivate the device limiting module; to put wireless network barrier, Bluetooth barrier, flight mode barrier, mobile data network barrier for the smart device (2); to display services and applications that can be seen by the users entering the password.

24. A system (1) according to claim 1, characterized by the central server (3) which is a server that can be accessed by the healthcare personnel (S) by means of the client device (5) and whereby s/he can view and set the data located in the central database (4) over thereof.

25. A system (1) according to claim 24, characterized by the central server (3) which can be accessed by the healthcare personnel (S) by means of the client device (5) and whereby s/he accesses the data located in the central database (4) over thereof and wherein the patient's (H) score, time, usage data related to the games and the tests are located.

26. A system (1) according to claim 24, characterized by the central server (3) which controls whether the healthcare personnel (S) has the data that s/he wants to access, view and set in the central database (4) or the necessary authorization about the patient (H) and the caregiver (B) with whom s/he wants to communicate.

27. A system (1) according to claim 26, characterized by the central server (3) which controls whether the healthcare personnel (S) descriptive information—that is matched with the patient (H) information entered by the caregiver (B) previously for controlling this authorization transaction—and the password information—that is required to be entered by the healthcare personnel (S)—are same as the ones entered by the healthcare personnel (S) by means of the client device (5).

28. A system (1) according to claim 1, characterized by the central server (3) which is configured such that it will be able to control the entries—that are made by the caregiver (B) by means of the smart device (2) and related to the type and the usage chart of the medications used by the patient (H)—automatically according to the information of medication unconformity and suitable usage chart included in the central database (4); and to send notification to the smart device (2) and/or the client device (5) in case of detecting an unconformity in the medication types related to the patient (H) or among the medications or detecting an unconformity in the control transaction according to the usage chart after this control.

29. A system (1) according to claim 28, characterized by the central server (3) which is configured such that it will be able to send medication reminders to the smart device (2) even if no data entry is made related to any medication type or a usage chart by the caregiver (B) by means of the smart device (2) based on the information of reference medication usage included in the central database (4) and prepared particularly for the patient (H) or the disease.

30. A system (1) according to claim 1, characterized by the central server (3) which is a server configured such that it will be able to send a reminder notification to the smart device (2) such that it also comprises the type and the level information of the game and the test, in cases where it is required to apply games and tests based on the information that are prepared previously, preferably by the healthcare personnel (S) about which games and tests should be applied to the patient (H) according to a what kind of timetable on the basis of the patient (H) and/or the disease and added to the central database (4).

31. A system (1) according to claim 1, characterized by the central server (4) wherein the web-based application enabling the healthcare personnel (S) to carry out all kinds of above-mentioned transactions by accessing over the client device (5) is installed.

32. A system (1) according to claim 1, characterized by the central server (4) wherein there are information of score, time, usage form related to the game and test transactions carried out by the patient (H) by means of the smart device (2); information for transactions of entering information about the patient (H) and the caregiver (B) carried out by the caregiver (B), creating a reminder record for appointment and social activity, making entry related to the type and the usage chart of the medications used by the patient (H), authorizing the healthcare personnel (S) who will take care of the patient (H); information about the patient (H), the caregiver (B), the smart device (2) and the healthcare personnel (S) that are separately descriptive and matched with each other; a password information based on the patient (H) about the healthcare personnel (S).

33. A system (1) according to claim 1, characterized by the central server (4) wherein there are usage chart data of medication conformity or unconformity based on the patient (H) or the disease.

34. A system (1) according to claim 1, characterized by the client device (5) which is a computer.

35. A system (1) according to claim 1, characterized by the client device (5) which is a smart phone that can provide access to the central server (3) and has capability to display the related graphic interfaces with respect to the transactions that can be carried out by the healthcare personnel (S).

36. A system (1) according to claim 1, characterized by the client device (5) which is a tablet computer that can provide access to the central server (3) and has capability to display the related graphic interfaces with respect to the transactions that can be carried out by the healthcare personnel (S).

37. A system (1) according to claim 34, characterized by the client device (5) which provides graphic interfaces—wherein transactions of: accessing the central server (3); viewing the data of patient (H) based game and test score, time, performance and usage data, the patient (H) information, the activity reminder records, the medication type and the usage chart information located in the central database (4) that is accessed by means of the central server (3); sending information and reminder notification (for example reminder notification for doctor's appointment) to the smart device (2) so as to be transmitted to the patient (H) and the caregiver (B); establishing written, voice and visual communication with the patient (H) and/or the caregiver (B) by means of the smart device (2)—can be carried out to the healthcare personnel (S).

38. A system (1) according to claim 34, characterized by the client device (5) which comprises camera, microphone and speaker.

39. A system (1) according to claim 1, characterized by the application server (6) which serves duties of sending device limiting commands and settings to the smart device (2) so as not to carry out any transaction except the transactions to be carried out by the patient (H) and the caregiver (B) on the smart device (2) in the management of the smart device (2) capabilities.

40. A system (1) according to claim 1, characterized by the application server (6) which also carries out the transactions of setting (adding, removing) and updating transactions related to the transactions to be carried out by the patient (H) and the caregiver (B) on the smart device (2), by means of the commands sent to the smart device (2).

41. A system (1) according to claim 1, characterized by the application server (6) which provides command communication among the smart device (2) over cloud messaging service.

42. A system (1) according to claim 1, characterized by the application server (6) which provides an interface for creating commands and settings to be sent to the smart device (2) and for sending these commands and settings.
